(19) United States
(12) Patent Application Publication
(10) Pub. No.: US 2003/0004471 A1
(43) Pub. Date: Jan. 2, 2003

(54) Needle protection device

(75) Inventors: Min-Hsiang Hung, Taichung (TW); Chao-Hsien Kuo, Taichung (TW)

Correspondence Address: BACON & THOMAS, PLLC, 625 SLATERS LANE, FOURTH FLOOR, ALEXANDRIA, VA, 22314

(73) Assignee: Sunder Biomedical Tech Co., Ltd., Taichung (TW)

(21) Appl. No.: 10/006,214

(22) Filed: Dec. 10, 2001

(30) Foreign Application Priority Data
Jun. 29, 2001 (TW) 090211007

(51) Int. Cl. A61M 5/00

(52) U.S. Cl. 604/263; 128/898

(57) ABSTRACT

A needle protection device includes a U-shaped first part for clamping a hose connected to the needle, and a second part connected to the first part. The body of a fluid or blood access device is inserted through the first part and two wings on the body are mounted on two sidewalls of the second part. Each of the sidewalls has a protrusion portion, a slide surface extending from the protrusion portion and a raised wall which is connected to the first end of the first part. A tunnel member extends from an inside of the second part and is located between the two sidewalls. The needle is oriented downward when the wings contact the raised walls and easily inserted beneath the tunnel member.

### FIELD OF THE INVENTION

The present invention relates to a needle protection device that includes a clamp portion and a needle protection portion which receives the needle beneath a tunnel member after the injection needle is used and the users will not hurt by the needle.

### BACKGROUND OF THE INVENTION

A conventional blood or fluid access device such as an A. V. F. is shown in FIG. 1 and generally includes a tubular body 15 which has a needle 11 on a first end of the body 15 and a fitting 17 on a second end of the body 15. The fitting 17 is connected to a hose through which medicine flows in the body 15 and the needle 11 and into the vessels of patients. A clamp device 113 is connected to the body 15 so that the fluid in the body 15 is stopped to flow to the needle when the clamp device 113 is turned to a close position. Two wings 13 extend from the body 15. A cap 111 is mounted to the body 15 and encloses the needle 11 so that the operators will not be hurt by the needle 11. However, for a nurse, during the frequent removal of the caps 111, it is possible that the fingers are hurt by the needle 11 and this could lead to a dangerous result for the nurse. Besides, the clamp device 113 and the cap 111 are two individual parts which are installed to the body 15 separately and are easily to get lost.

### SUMMARY OF THE INVENTION

In accordance with one aspect of the present invention, there is provided a needle protection device which comprises a U-shaped first part having a base portion and a pushing portion. A gap is defined between the base portion and the pushing portion. The pushing portion has a first protrusion extending from a surface of the pushing portion that faces the base portion, and the base portion has a second protrusion extending from a surface of the base portion that faces the pushing portion. A first hole and a second hole are respectively defined through two ends of the first part. A second part extends from the first end of the first part and two sidewalls extend from two sides of the second part. Each of the sidewalls has a protrusion portion, a slide surface extending from the protrusion portion and a raised wall which is connected to the first end of the first part. The first hole communicates with a space between the two sidewalls. A tunnel member extends from an inside of the second part and is located between the two sidewalls. The tunnel member has an opening defined in one end thereof and a close end at the other end of the tunnel member.

The primary object of the present invention is to provide a needle protection device that has a clamp portion for clamp a duct and a needle protection portion that receives the needle beneath a tunnel member without touching the needle.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a plan view to show a conventional A. V. F.;

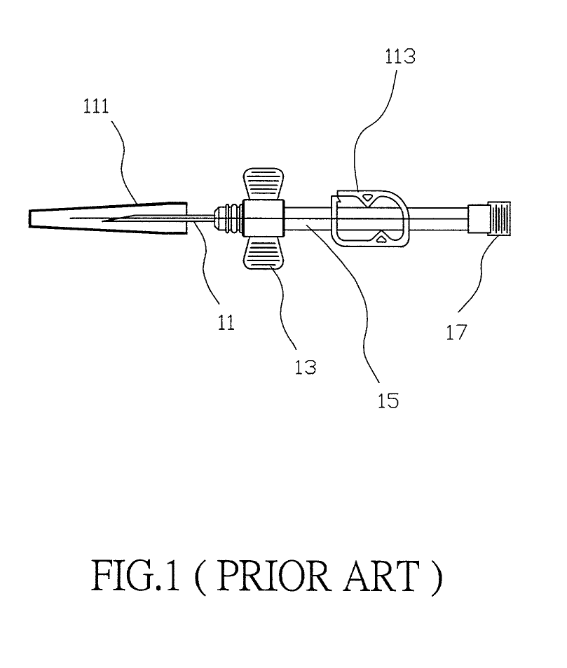

FIG. 2 is a perspective view to show the needle protection device of the present invention;

FIG. 3a is a side view to show that an A. V. F. is engaged with the needle protection device of the present invention;

FIG. 3b is a side view to show that the A. V. F. is slid along two sidewalls of the needle protection device of the present invention;

FIG. 3c is a side view to show that the wings contact the raised walls and the needle is oriented downward;

FIG. 3d is a side view to show that the needle is moved beneath the tunnel member via the slot of the needle protection device of the present invention, and

FIG. 4 is a perspective view to show the A. V. F. is engaged with the needle protection device of the present invention.

### DETAILED DESCRIPTION OF THE INVENTION

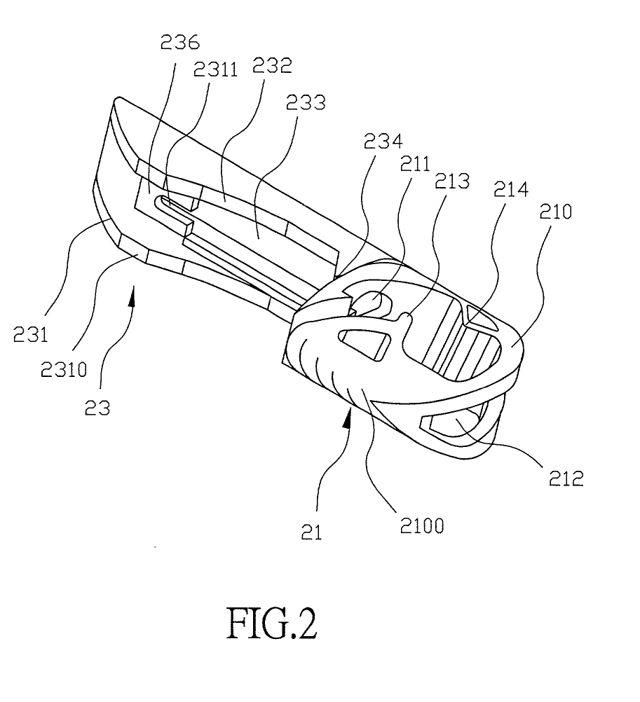

Referring to FIG. 2, the needle protection device of the present invention comprises a U-shaped first part 21 and a second part 23, wherein the first part 21 is used as a clamping portion and has a base portion 210 and a pushing portion 2100. A gap is defined between the base portion 210 and the pushing portion 2100 so that the pushing portion 2100 is able to be pushed toward the base portion 210. The pushing portion 2100 has a first protrusion 213 extending from a surface of the pushing portion 2100 that faces the base portion 210, and the base portion 210 has a second protrusion 214 extending from a surface of the base portion 210 that faces the pushing portion 2100. A first hole 211 and a second hole 212 are respectively defined through two ends of the first part 21.

The second part 23 is used as a needle protection portion and extends from the first end of the first part 21; and two sidewalls 231 extend from two sides of the second part 23. Each of the sidewalls 231 has a protrusion portion 2310 on a top edge thereof and a slide surface 232 extends from the protrusion portion 2310 along the top edge of each of said sidewalls. A raised wall 234 connected to the slide surface 232 is connected to the first end of the first part 21. The first hole 211 communicates with a space 233 between the two sidewalls 231.

A tunnel member 236 extends from an inside of the second part 23 and is located between the two sidewalls 231. The tunnel member 236 has an opening an one end thereof and a close end at the other end of the tunnel member 236. A slot 2311 is defined in a top of the tunnel member 236 and communicates with the opening.

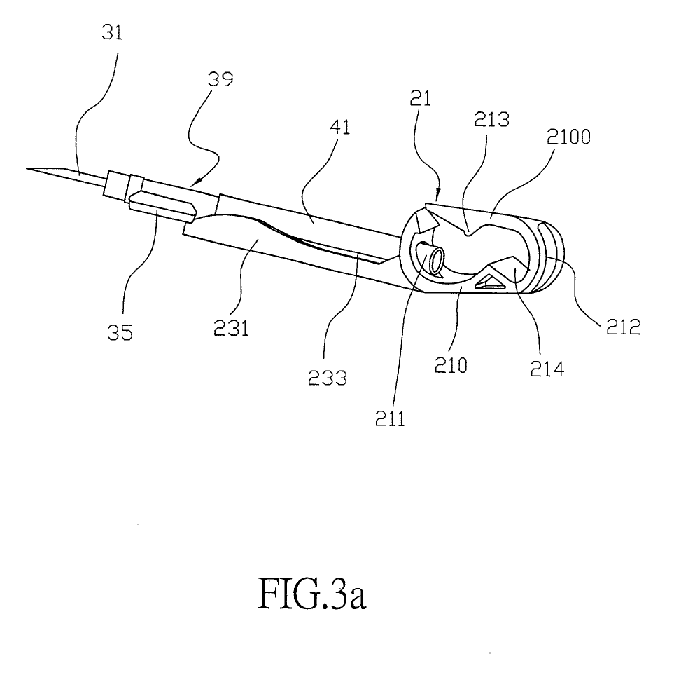

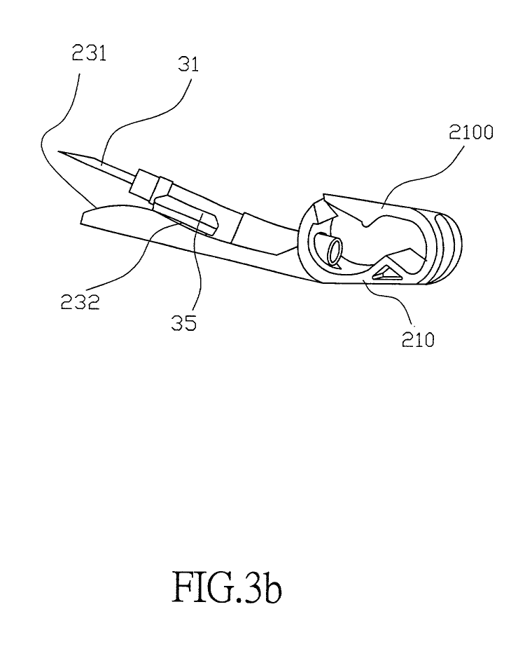

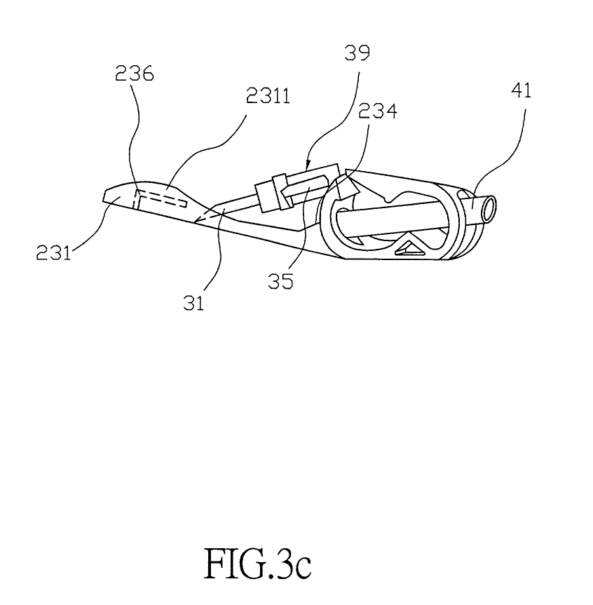

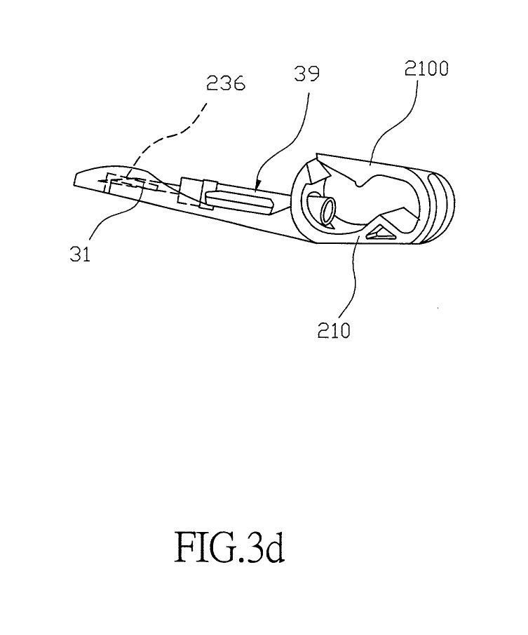

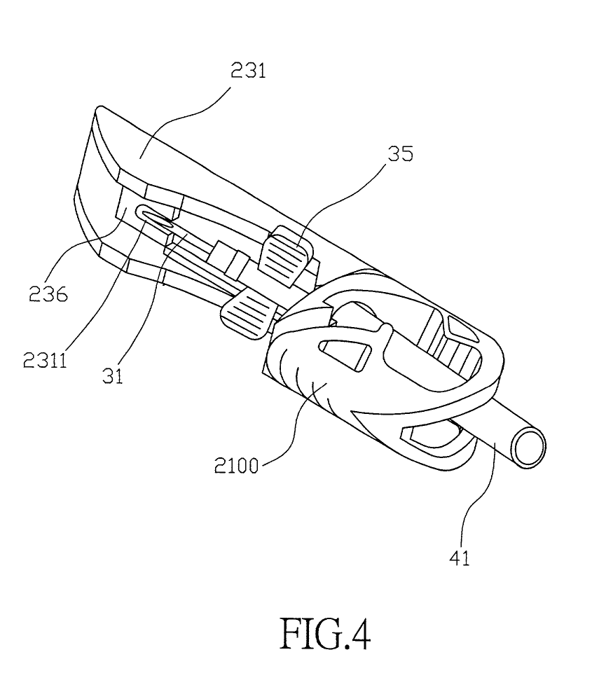

Referring to FIGS. 3a to 3d and 4, an A. V. F., for instance, is inserted in the first part 21 via the second hole 212 and the first hole 211, and enters in the space 233 between the two sidewalls 231 of the second part 23. A hose 41 connected to the A. V. F. is located between the first protrusion 213 and the second protrusion 214 so that when pushing the pushing portion 2100, the hose 41 is clamped by the two protrusions 213, 214. The body 39 of the A. V. F. is located between the two sidewalls 231 and the two wings 35 are mounted on the two sidewalls 231. When using the needle 31, the device is pulled away from the needle 31 to allow the needle 31 extend from the second part 23 as shown in FIG. 3a. When the injection is finished, the device is pushed toward the needle 31 and the wings 35 are moved along the slide surfaces 232 of the sidewalls 231 till the wings 35 contact the raised walls 234 as shown in FIGS. 3b and 3c. The needle 31 is oriented downward when the wings 35 contact the raised walls 234 so that the needle 31 is easily moved beneath the tunnel member 236 via the slot 2311 as shown in FIGS. 3d and 4 by slightly pulling the device.

The needle 31 is enclosed by the sidewalls 231 and the tunnel member 236 so that the users do not touch the needle 31. The needle 31 can be enclosed by the tunnel member 236 and the sidewalls 231 so that the users do not need to re-cap the cap onto the needle 31. The user holds the device and does not touch the needle 31 during the processes of moving the needle 31 to the tunnel member 236 so that the user is protected well by the device of the present invention.

While we have shown and described the embodiment in accordance with the present invention, it should be clear to those skilled in the art that further embodiments may be made without departing from the scope of the present invention.

What is claimed is:

1. A needle protection device comprising:
a first part adapted to clamp a hose connected to a needle;
a second part extending from a first end of said first part and two sidewalls extending from two sides of said second part each of said sidewalls having a protrusion portion defined in a top edge thereof, a slide surface extending from said protrusion portion along said top edge of each of said sidewalls and a raised wall connected to said slide surface, said raised wall connected to said first end of said first part, and
a tunnel member extending from an inside of said second part and located between said two sidewalls, said tunnel member having an opening defined in one end thereof and a close end at the other end of said tunnel member.

2. The device as claimed in claim 1, wherein said tunnel member has a slot defined in a top thereof and said slot communicates with said opening.

3. The device as claimed in claim 1, wherein said first part is a U-shaped member which has a base portion and a pushing portion, a gap defined between said base portion and said pushing portion, said pushing portion having a first protrusion extending from a surface of said pushing portion that faces said base portion, said base portion having a second protrusion extending from a surface of said base portion that faces said pushing portion, a first hole and a second hole respectively defined through two ends of said fist part said first hole communicating with a space between said two sidewalls of said second part.
